(19) United States
(12) Patent Application Publication
(10) Pub. No.: US 2025/0321228 A1
(43) Pub. Date: Oct. 16, 2025

(54) MICROIMPRINTING OF ANTIBODIES AND BIOMOLECULES FOR CELL PHENOTYPING AND ACTIVATION

(71) Applicants: Institut National de la Santé et de la Recherche Médicale, PARIS (FR); Université d'Aix-Marseille, MARSEILLE (FR); Centre National de la Recherche Scientifique, PARIS (FR)

(72) Inventors: Olivier THEODOLY-LANNES, Marseille (FR); Philippe ROBERT, Marseille (FR); Geoffrey DELHAYE, Marseille (FR)

(21) Appl. No.: 18/866,961

(22) PCT Filed: May 17, 2023
(86) PCT No.: PCT/EP2023/063407

§ 371 Date: Nov. 18, 2024

(30) Foreign Application Priority Data
May 18, 2022 (EP) 22305735.7
Jun. 30, 2022 (EP) 22305954.4

(51) Int. Cl. G01N 33/543; G01N 33/68

(52) U.S. Cl. CPC G01N 33/54386; G01N 33/6854; G01N 2333/7051; G01N 2333/70514; G01N 2333/70517; G01N 2333/7056; G01N 2333/70596

(57) ABSTRACT

The device comprises a substrate comprising a first zone (1) on which is absorbed a protein capable of binding to a first membrane molecule and comprising a second zone (2), on which an antibody is absorbed, targeting a second membrane molecule, the first zone and the second zone extending together in length over a dimension comparable to the length of a cell.

### CROSS-REFERENCE TO RELATED APPLICATIONS

The present application is a filing under 35 U.S.C. 371 as the National Stage of International Application No. PCT/EP2023/063407, filed May 17, 2023, entitled “MICROIMPRINTING OF ANTIBODIES AND BIOMOLECULES FOR CELL PHENOTYPING AND ACTIVATION,” which claims priority to European Application No. 22305735.7 filed with the European Patent Office on May 18, 2022 and claims priority to European Application No. 22305954.4 filed with the European Patent Office on Jun. 30, 2022, all of which are incorporated herein by reference in their entirety for all purposes.

### TECHNICAL FIELD

The invention generally relates to the field of quantifying the expression of membrane molecules and cellular functions.

The invention relates more particularly to a functional test for clinical immunology.

### PRIOR ART

In biological science and medicine, it is often necessary to identify cell types and quantify their functions. In particular, the presence of specific molecules in the cell membrane is a traditional, effective means of identification. Taking the example of immunity, which is strongly implicated in most diseases, the rapid and accurate quantification of human immune functions is of great importance for the early detection of the development of an infectious pathology or the outcome of an organ transplant, the choice of treatments and the monitoring of their efficacy.

Knowledge of the immune system's early responses to a pathogen is therefore essential for diagnosis and the therapeutic application of appropriate treatments.

Various techniques for quantifying lymphocyte functions, including flow cytometry, lymphocyte proliferation, and cytokine production, are thus known from the prior art.

Also known from the prior art is the micrometric printing of proteins on a dirt-repellent substrate (LIMAP technology) and reflection interference microscopy (RIM) technology, which enables adhesion zones between micrometric transparent objects to be visualized.

However, rapid visualization of immune system responses is, for the prior art, a difficult problem, even though a long-standing need exists in this area.

For the purposes of the present application, a “CD45 antibody” refers to the antibody targeting the marker designated “CD45” in the nomenclature of differentiation classes or clusters. In general, a “CDx antibody”, where x is a natural number, such that CDx belongs to the nomenclature of differentiation clusters, designates an antibody targeting the family of membrane glycoproteins or marker or antigen or membrane antigen designated by “CDx”.

For the purposes of this application, membrane protein for a cell means any glycoprotein present on the external surface of the cell and capable of forming a bond with a specific antibody, in particular:
CD3 is a membrane protein as described in the paper “A novel Leukocyte Adhesion Deficiency III variant: Kindlin 3 defect results in integrin and non-integrin related defects in different steps of leukocyte adhesion” (Philippe Robert, J Immunol May 1, 2011, 186 (9) 5273-5283; DOI: https://doi.org/10.4049/jimmunol.1003141)
CD4 is a membrane protein as described in “A microfuidic device for practical label-free CD4+ T cell counting of HIV-infected subjects.” (Xuanhong Cheng-Lab on a chip 2007, Volume 7 Issue 2 170-178 doi: 10.1039/b612966h).

If the antibody is deposited on a substrate, the membrane protein will be a substrate adhesion membrane protein, the adhesion in question being measured by the energy of the antigen-antibody bond and of such a nature as to keep the cell at a distance from a substrate equal to the length of the antigen-antibody bond and to have a detectable optical effect.

The energy of antigen-antibody binding is generally measured by the terms “affinity” or the stability of binding by “avidity” for multivalent antigens and antibodies.

For the purposes of this application, “antibody” means both a specific and a cross-reactive antibody, whether monovalent or multivalent; that is any known antibody capable of being deposited on a biocompatible substrate and binding to a membrane protein.

In particular, in cases involving chimeric or other proteins, the name “antibody” in the sense of the present application may be understood to refer to molecules which are not designated an “antibody” in the prior art, but which are able to assemble with another molecule via a protein domain originating from an antibody, for example in the case of CAR T lymphocytes which adhere to their ligand via a chimeric antibody domain.

The antigen-antibody bonds considered in the present application are those which keep the antigen fixed in the antibody site and which, according to the prior art, are non-covalent in nature. These include hydrogen bonds, electrostatic bonds, Van der Waals forces, and hydrophobic bonds. Multiple bonds between the antigen and the antibody ensure a stable interaction between these two molecules, and those considered in the present application are those which enable the cell to be physically kept at a fixed distance from a substrate, and therefore to use physical means to detect the presence of the cell in the vicinity of the substrate, notably optically.

The terms “optical interference reflection” or “reflection interference contrast microscopy” refer to the technique known by the acronym IRM (Interferential Reflection Microscopy).

### GENERAL OVERVIEW

In this context, the present application relates to a device which comprises a substrate comprising a first zone on which is adsorbed a protein capable of binding to a first membrane molecule and comprising a second zone on which an antibody is absorbed, targeting a second membrane molecule, the first zone and the second zone extending together in length over a dimension comparable to the length of a cell.

Advantageously, the present application relates to a device characterized in that it comprises a substrate, the substrate comprising a first zone on which is adsorbed a protein capable of specifically binding to a cell by interacting with a first membrane molecule and comprising a second zone on which is adsorbed an antibody targeting a second membrane molecule expressed on the surface of said cell,
said first zone representing a first surface defining a first zone A1,
said second zone representing a second surface, defining a second zone A2,
where the total area represented by the sum of areas A1 and A2 is less than or equal to the area of the projected surface of said suspended cell (FIG. 1).

In the context of the present invention, the device comprises two zones which are organized so that a cell can interact with the first zone, or the second zone, or both zones simultaneously. The aim is to detect the overlap of the first zone or the second zone or both zones and thus to phenotype the determined cell by virtue of the protein and antibody both adsorbed on the first and second zone respectively.

To enable overlap detection, the first and second zones have a particular configuration so that the total area corresponding to the sum of the area of the first and second zones is less than or equal to the area of the projected surface of said cell.

Thus, if said cell is able to recognize the protein adsorbed on the first zone, said cell will adhere to it, forming an optical microscope contrast by reflection interference contrast in the first zone which will appear dark, the second zone remaining light-colored in appearance (FIG. 2B). If, moreover, said cell having interacted with the first zone is capable of interacting with the antibody adsorbed on the second zone, the cell adhering to the first zone will spread out over the second zone to adhere to it, forming an optical microscope contrast due to reflection contrast (which is one possible way of revealing the signal generated by the device) in the second zone, which will also appear dark (FIG. 2C). In the absence of cell adhesion to the device, the entire pattern remains light-colored. (FIG. 2A, FIG. 3). The projected surface of a suspended cell is taken to mean the plane defined by the largest dimensions of a cell when viewed from above. For example, if the cell is a perfect sphere, the projected surface will be a disk of radius r, r being the radius of the perfect sphere. The projected surface will then have an area of πr² and the total area (A1+A2) will be less than πr². Length is the largest dimension of a two- or three-dimensional geometric shape (as opposed to width or height).

A two-dimensional shape has an area less than or equal to its squared length; a three-dimensional shape has a projected area less than or equal to its squared length.

As the device can be produced in different geometric shapes, it is defined in the invention in terms of the length of the cells with which the device is likely to interact. In other words, the device and the zones it contains will be defined according to the cell under consideration.

Cells in suspension correspond either to spontaneously non-adherent cells (blood or hematopoietic cells), or to adherent cells that have been detached from their support (e.g. by trypsin treatment).

In variants (FIG. 4):
the first membrane molecule is a membrane molecule common to a first cell type and wherein the second membrane molecule is a membrane molecule common to a subtype of the first cell type.
the first membrane molecule is a membrane molecule common to a first cell type and wherein the second membrane molecule is a membrane molecule common to a particular state of the first cell type.
the first membrane molecule is a membrane molecule common to a first cell type and wherein the second membrane molecule is a membrane molecule common to a second cell type capable of interacting with the first cell type.
the protein is an antibody targeting CD4 and the antibody is an antibody targeting CD8.
the protein is an antibody targeting CD3.
the protein is CD19.
the antibody targets CD69.
the antibody targets CD25.
the antibody targets CD107.
the antibody is an anti-collagen antibody.
the antibody targets CD86.

Advantageously, the device enables a cell to be identified via the protein adsorbed on the first zone, and the antibody adsorbed on the second zone also enables the cell to be identified, for example to confirm its phenotype. An example of this device comprises, adsorbed on the first zone, a protein which is an antibody targeting the CD3 antigen, and the antibody adsorbed on the second zone is an antibody targeting any of the CD4 or CD8 antigens, or a mixture thereof.

Advantageously, the device enables a cell to be identified via the protein adsorbed on the first zone, and the antibody adsorbed on the second zone to activate the cell, for example to induce a new cell function or cell differentiation. Examples of such a device are:
the protein adsorbed on the first zone is an antibody targeting the CD45RO antigen and the antibody adsorbed on the second zone is an antibody targeting the CD3 antigen, or a mixture of antibodies targeting the CD3 antigen and the CD28 antigen, or
the protein adsorbed on the first zone is an antibody targeting the CD56 or CX3CR1 antigen and the antibody adsorbed on the second zone is an antibody targeting the CD20 antigen (rituximab), or
ii—the protein adsorbed on the first zone is an antibody targeting the CD14 antigen and the antibody adsorbed on the second zone is an antibody targeting any of the CD16, CD32 or CD64 markers, or targeting membrane lipopolysaccharides (LPSs).

Advantageously, the device enables a cell to be activated via the protein adsorbed on the first zone, and the antibody adsorbed on the second zone to detect a cellular response in the form of expressed membrane molecules, such as activation or senescence, that is to read this response. Examples of such a device are:
the protein adsorbed on the first zone is CD19 or a chimeric protein comprising CD19 and the antibody adsorbed on the second zone is an antibody targeting any of the antigens CD69, CD107, CD25, CD57, TIM-3 and LAG-3, or
the protein adsorbed on the first zone is an antibody targeting the CD20 antigen (rituximab) and the antibody adsorbed on the second zone is an antibody targeting the CD107 antigen.

Advantageously, the device enables a cell to be identified via the protein adsorbed on the first zone, and the antibody adsorbed on the second zone to detect a cellular response in the form of expressed membrane molecules, such as activation or senescence, that is to read this response. One example of such a device is such that the protein adsorbed on the first zone is CD19 and the antibody adsorbed on the second zone is an antibody targeting any of the antigens CD69, CD107, CD25, CD57, TIM-3 and LAG-3.

Also described is a device as defined above, wherein the first membrane molecule is a membrane molecule expressed at the surface of a first cell type and wherein the second membrane molecule is a membrane molecule expressed at the surface of a subtype of said first cell type, said subtype of said first cell type expressing at its surface said first and said second membrane molecule.

The present application further relates to a method which comprises the following steps:
adsorbing on a substrate a protein capable of binding to a first membrane molecule, in a first subcellularly-dimensioned zone of the substrate,
adsorbing an antibody targeting a second membrane molecule onto a substrate, in a second subcellularly-dimensioned zone of the substrate.

The present application also relates to a variant of the method for quantifying and monitoring cell activation kinetics, which comprises the following steps:
adsorbing onto a substrate a protein capable of binding to a first membrane molecule common to a first cell type, in a first subcellularly-dimensioned zone,
adsorbing onto a substrate an antibody targeting a second membrane molecule common to a particular state of the first cell type, in a second subcellularly-dimensioned zone,
bringing a population of cells of the first type comprising cells of the first type allowing activation kinetics towards a particular state into contact with the substrate,
detecting the presence of a cell of the first type in the first zone using optical microscopy,
quantifying the kinetics of cell activation of the first type by detecting cell adhesion of the first type in the second zone, using optical microscopy.

The present application also relates to a variant of the method for selecting a cell type and for triggering its activation, which comprises the following steps:
adsorbing onto a substrate a protein capable of binding to a first membrane molecule common to a first cell type, in a first subcellularly-dimensioned zone,
adsorbing onto a substrate an antibody targeting a second membrane molecule known to trigger the activation of the first cell type, in a second subcellularly-dimensioned zone,
bringing a population of cells comprising cells of the first type allowing an activation towards a particular state into contact with the substrate,
detecting the presence of a cell of the first cell type expressing the first membrane molecule in the first zone, using optical microscopy,
detecting the triggering of the cell activation of the first type by detecting cell adhesion of the first type in the second zone, using optical microscopy.

The present application also relates to a variant of the method for detecting cell membrane molecules, which comprises the following steps:
adsorbing onto a substrate a protein capable of binding to a first membrane molecule common to a first cell type, in a first subcellularly-dimensioned zone,
adsorbing onto a substrate an antibody targeting a second membrane molecule common to a subtype of the first cell type, in a second subcellularly-dimensioned zone,
bringing a population of cells of the first type comprising cells of the subtype into contact with the substrate,
detecting the presence of a cell of the first cell type expressing the first membrane molecule in the first zone, using optical microscopy,
quantifying the cell expression of the second membrane molecule by detecting cell adhesion in the second zone, using optical microscopy.

The present application also relates to a variant of the method for detecting an interaction of two cells, which comprises the following steps:
adsorbing onto a substrate a protein capable of binding to a first membrane molecule common to a first cell type, in a first subcellularly-dimensioned zone,
adsorbing onto a substrate an antibody targeting a second membrane molecule common to a second cell type capable of interacting with the first cell type, in a second subcellularly-dimensioned zone,
bringing a population of cells comprising cells of the first type and cells of the second type into contact with the substrate,
detecting the presence of a cell of the first type in the first zone using optical microscopy,
detecting the presence of a cell of the second type or quantifying the expression of the second membrane molecule by the second cell in the second zone using optical microscopy.

The use of a substrate, particularly in vitro, to characterize the activation or differentiation state of a given cell in a cell population is also described,
said substrate comprising a first zone on which is adsorbed a protein able to bind specifically to said determined cell by interacting with a first membrane molecule specific to said determined cell and comprising a second zone on which is adsorbed an antibody targeting a second membrane molecule expressed on the surface of said determined cell,
said first zone representing a first surface defining a first zone A1,
said second zone representing a second surface, defining a second zone A2,
where the total area represented by the sum of areas A1 and A2 is less than or equal to the area of the projected surface of said suspended cell,
said second membrane molecule being expressed on the surface of said specific cell when said cell is activated or differentiated.

Generally speaking, the invention is based on a method that enables phenotypic and single-cell analysis of the activation properties of immune cells, as shown in FIGS. 1 to 5

The method can use optical microscopy to generate multiple micro-zones (micropatterns) of different proteins and subcellular sizes (FIG. 3), and these microengineered substrates are capable of performing multiple functional assays on immune cell suspensions or whole blood, such as (i) selecting/identifying a cell type of interest, (ii) triggering an activation signal, and (iii) reading the kinetics of immune activation. The invention is not limited to such optical microscopy, and any instrument projecting ultraviolet rays (e.g. a confocal microscope) or any (micro-)printing technique can also be used. The person skilled in the art will be able to determine the most appropriate instrumentation.

These functions are assessed by the nature of the micropattern proteins, which are generally antibodies with specific affinities and effective actions. When the cell membrane of the suspension expresses the antibody target on the substrate, the cell spreads over the corresponding pattern (FIG. 2) and the cell adhesion imprint can be detected by interferometric microscopy (FIG. 2). Detection is not limited to reflection interference contrast imaging, and detection by transmission imaging and image collection to detect cell contours can also be used. Here again, the person skilled in the art will be able to determine which technique is the most appropriate.

The different functions performed by the micropattern can be evaluated by taking a single image and analyzing the spread of cells in each pattern. It's important to note that the readout requires none of the tedious manipulations usually involved in immunolabeling-based techniques (cell preparation, incubations, rinsing). In a typical interference microscopy image, dark zones indicate adhesion and light zones non-adhesion (FIGS. 2 and 3). In practice, the operator's tasks are limited to placing the smart substrate on a dedicated optical microscope and depositing the cell sample on the substrate.

### BRIEF DESCRIPTION OF THE FIGURES

FIGS. 1A-1B: in A, an example of a printed pattern shown in plan view at the top, and in cross-section at the bottom. The pattern comprises two concentric zones (1 and 2) each carrying an antibody of a particular specificity, in the example CD3 in the center (1) and CD69 in the periphery (2). In B, an example of a circulating cell (a T lymphocyte) shown at the same scale, with a membrane marker on its surface (CD3 here), which is likely to interact with the central zone of the pattern. In this example, the largest dimension of the pattern (10 μm) is smaller than that of the cell (12 μm).

FIGS. 2A-2C: examples of different cells interacting with a pattern identical to that shown in FIG. 1, with a side view at the top and a plan view at the bottom of the signal obtained in reflection interference contrast (which is one possible way of revealing the signal generated by the device). In A, an example of interaction between a cell carrying no membrane marker corresponding to the specificities of the antibodies present. A B lymphocyte (whose CD20 marker is shown) does not express a membrane ligand to interact with the pattern's antibodies and does not adhere to the surface; the resulting appearance in reflection interference contrast is an absence of signal. In B, an example of interaction between a cell carrying a single type of membrane marker corresponding to one of the specificities of the antibodies present on the pattern. A non-activated T lymphocyte expresses CD3 but not CD69. Its CD3 molecules are captured by antibodies in the central zone of the pattern, and its membrane therefore adheres to this zone. This creates an interference contrast in the central zone of the pattern. In C, an example of interaction between a cell carrying the two types of membrane markers corresponding to the specificities of the two types of antibody present on the pattern. Its CD3 molecules are captured by antibodies in the central zone of the pattern, and its CD69 molecules are captured by antibodies in the peripheral zone of the pattern, so its membrane adheres to both zones. This creates an interference contrast in the central zone and peripheral zone of the pattern.

FIGS. 3A-3C: examples of real images obtained during an experiment using the type of pattern described in FIGS. 1 and 2, repeated to form a matrix of patterns on a glass slide. In A, a transmission optical microscope image, showing T lymphocytes each adhered to a matrix pattern location. Some locations did not capture any cells. In B, a reflection interference contrast image at the same magnification showing that each cell adhering to a pattern in A produces contrast on the central zone of the pattern in B, identifying each cell as expressing CD3 on its surface and therefore being a T cell. Some of the cells produce contrast to varying degrees on the peripheral zone of the pattern to which they adhere, identifying these cells as expressing CD69 on their surface and therefore being activated T cells. In C, a fluorescence microscopy image of the same sample at the same magnification, after the cells have been fixed with formaldehyde and incubated with a fluorescent anti-CD69 antibody, showing the membrane expression of the molecule and allowing us to observe the matching alignment with the contrast produced on the peripheral zone of the patterns for each cell: the bright cells in C generally occupy the peripheral zone of the patterns.

FIG. 4: examples of possible patterns with two zones, each carrying a type of antibody with a particular specificity (identical to the patterns described in FIGS. 1 and 2). Example of a pattern with three zones, each carrying a type of antibody with a particular feature. Total pattern dimensions remain below 10 μm, which is less than the suspension dimension of the targeted cell type (here, leukocytes).

FIG. 5: example of a pattern with three zones, each carrying a type of antibody with a particular feature. Total pattern dimensions remain below 10 μm, which is less than the suspension dimension of the targeted cell type (here, leukocytes).

### DETAILED DESCRIPTION OF EXAMPLE(S)

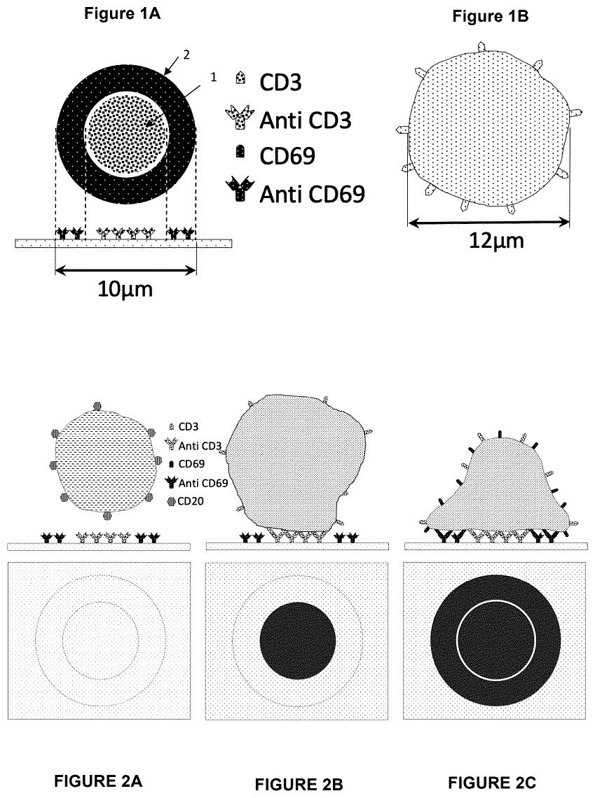

In a first embodiment, with reference to FIG. 1 for the numbers of the elements in bold or the elements in brackets, the invention can be carried out for the substrate by means of LIMAP technology, which enables proteins to be aligned and adsorbed on substrates coated with a PEG (Polyethylene Glycol) brush insulated according to an ultraviolet light pattern, thereby achieving specific adhesion of a protein in the insulated zones.

However, in each case where the adhesion of several proteins is required, and in particular for two antibodies, it is necessary to be able to specifically deposit the first antibody and then the second, each specifically on the desired pattern.

Once a specific process has been developed for a pattern suitable for a single cell, it is possible to spatially duplicate the production of multiple patterns in parallel to rapidly obtain a pattern matrix enabling tests to be carried out on as many captured cells as there are patterns present on the LIMAP-printed substrate.

Experimentally, a first zone (1) is locally printed by the combined action of a photoinitiator (PLPP, Alveole), placed in solution on a PEG-SVA brush and subjected to a projection of an ultraviolet pattern at 375 nm (step 1). A first antibody is then incubated for 12 hrs at 4° C. (step 2). A second zone (2) is then printed by LIMAP (step 3). A second antibody is added and incubated for 12 hrs at 4° C. (step 4).

In this context, the second antibody adsorbs non-specifically onto the second zone (that is not limited to the most recently illuminated pattern), as it is also adsorbed in the first zone. The anti-fouling substrate (PEG brush) is rendered adhesive in the first zone by the first illumination and in the second zone by the second illumination, which means that the second antibody adsorbs not only in the second zone but also in the first zone. These properties can be verified by epifluorescence, for example, using separate fluorescence markers for the antibodies and observing them with interference filters that isolate the wavelength of each fluorescence marker.

To avoid non-specific adsorption of the second antibody in the first zone, the person skilled in the art may use passivation techniques known in the prior art, such as the use of passivation solutions common in biophysics, between the two illumination phases, in particular:
4% BSA in PBS for 1 hr at room temperature,
4% BSA in PBS for 30 min followed by 1 mg/mL rabbit serum IgG for 30 min at room temperature,
1% Pluronic F127 in PBS for 15 min at room temperature

The best passivation solution found was PEG-SVA at 0.23 mg/mL with 10 mM sodium bicarbonate in Milli-Q water for 15 min at room temperature.

Once the passivation method has been chosen, based on minimal non-specific adsorption, LIMAP technology can be used to create a periodic pattern comprising two potential cell adhesion zones: one specific for T lymphocytes as a whole, and one for activated T lymphocytes. It will also be possible to create as many adhesion zones as required by illuminating each zone, exposing it to an antibody chosen for the zone, then passivating and illuminating the next zone, and thus so on.

Conveniently, for each zone, the zone taken as the pattern of a spatially periodic structure is duplicated in a spatially periodic manner, shifting the pattern on the substrate to enable deposition of the same antibody and passivation of all illuminated patterns in a single step.

Duplication is conveniently carried out in a known way using LIMAP technology, in which an array of micro-mirrors is used to produce the periodic pattern and is imaged onto the substrate in ultraviolet light, all at once.

Thus, each antibody is adsorbed only in its zone, that is specifically in this zone, the antibodies are adsorbed in contiguous zones forming a pattern whose length, meaning the largest geometric dimension, is comparable that is less than or equal to the length (again meaning the largest geometric dimension) of the cell to be adhered to, and with patterns spaced apart so that only one cell can adhere to each pattern. The person skilled in the art can adapt the shape and length of each zone and the distance between patterns by simple operations to ensure that, for a particular cell type, only one cell adheres per pattern and in a given order of adhesion or a given spatial configuration of adhesion.

For example, for phenotyping memory T lymphocytes, a first adhesion zone of the aCD45RO type (“antibody targeting CD45RO”) delimited inside a circle and a second adhesion zone of the aCD69 type (“antibody targeting CD69”) delimited on a circular ring concentric with the first zone.

For example, to monitor the activation of memory T lymphocytes, a first adhesion zone of the aCD45RO type (“antibody targeting CD45RO”) mixed with aCD3 and aCD28 is chosen to make all memory T lymphocytes adhere (all expressing CD45RO) and then trigger the activation of cells that have adhered (by CD3+CD28), the first zone being further delimited within a circle and the same second aCD69-type adhesion zone (“antibody targeting CD69”) delimited on a circular ring concentric with the first zone to adhere only activated memory T lymphocyte cells specifically expressing CD69.

The device of the invention can thus be versatilely used either statically for phenotyping activated memory T lymphocytes, or dynamically for monitoring activation of naive memory T lymphocytes by adding to an adhesion antibody common to all memory T lymphocytes, a memory T lymphocyte antibody.

Such a device can be easily adapted to any type of cell that can be activated.

In this embodiment, a substrate is thus obtained on which T lymphocytes can progress and move, until they attach to each first zone of the periodically repeated pattern and adhere, only if activated, to each second zone of the periodically repeated pattern.

It will be possible to vary the maximum size of the first zone and/or the second zone, by performing zone tests of variable illuminated diameter on a substrate. This size is of the order of a few micrometers, for example from 4 μm to 12.5 μm to induce the adhesion of a single immune cell.

As already mentioned, the CD45 antibody or a CD45/CD3/CD28 antibody mixture can be deposited in each first zone, as long as it induces non-specific adhesion of any T lymphocyte, activated or non-activated, in particular memory T lymphocytes.

For example, in each second zone, the CD69 antibody or a mixture of antibodies containing it, such as CD69+CD25, or a third contiguous zone containing CD25, can be deposited, as long as it induces the specific adhesion of any activated T lymphocyte, particularly memory T lymphocytes. It is also possible to deposit only CD25, to observe secondary T lymphocyte activation rather than primary activation (that is temporally preceding secondary) with CD69, or both with CD69+CD25.

The activation kinetics can be tracked in a known way using MRI microscopy, with light zones revealing non-adhesion, implying that non-activated T lymphocytes will have a first dark zone and a second zone that is brighter the weaker their adhesion, the greater their distance from the substrate, or the weaker their activation.

The shape of the zone printed, deposited or coated with an antibody may be contained within a circle or ring.

For example, a suitable pattern for the first zone is a disk, and a suitable pattern for the second zone is a ring concentric with the disk, both with a maximum dimension of around ten microns.

Observation of T lymphocyte adhesion to the substrate in the first and second zones then enables the enumeration of early-activated lymphocytes and kinetic monitoring of their activation intensity as a function of their adhesion intensity. A camera recording thus enables precise, quantitative monitoring of kinetics at an early stage of T lymphocyte activation, helping to deliver rapid results.

The interpretation of the invention is particularly straightforward, since adhesion to the ring of a pattern is synonymous with activation of the T lymphocyte captured on the pattern, and the absence of adhesion to the ring is characteristic of a non-activated T lymphocyte. Using MRI as a method of obtaining contrast, for example, the appearance of the ring of a pattern is synonymous with activation of the T lymphocyte captured on the pattern, and the absence of the ring appearing is characteristic of a non-activated T lymphocyte.

Using visible optical markers, it will be possible to determine for each site containing a first and second pattern, whether a T lymphocyte is present and whether it is activated, without recourse to fluorescence and therefore without manipulation.

Numerous variants are possible on the general inventive principle of the invention, which is to deposit, in a first micrometric zone, a first antibody allowing the adhesion of any T lymphocyte, activated or non-activated (that is an antibody not specific to activated T lymphocytes) and then to deposit, in a second micrometric zone, a second antibody allowing the adhesion of any activated T lymphocyte (that is an antibody specific to activated T lymphocytes) The invention therefore makes it possible to monitor the activation of T lymphocyte immune functions in real time, from the very start of an infection or transplant.

For B lymphocytes, the CD20 antibody can be used instead of the CD45RO antibody.

For a given family of cells not specifically expressing CDx and specifically expressing CDy (where y is different from x) in a particular state, it will thus be possible to deposit contiguously for a cell (in the sense of extending over a length of a cell) that is over a cell domain, aCDx and aCDy to detect cells in the particular state or cells progressing towards the particular state, by additionally depositing aCDz, where CDz is expressed upon the activation of the particular state. As each cell can occupy at most one domain, or each domain can accommodate at most one cell, it will be possible to perform quantitative and qualitative techniques on cell populations according to the teaching of the present application.

The geometries of the zones described are non-limiting and, in particular, non-concentric zones may be taught in the present application for certain types of cells.

The present application thus describes a widely available and easily adaptable tool for studying various cells and their states, quantitatively and qualitatively, with a test practicable with only an MRI-equipped microscope for observing the results and conventional means of preparing cells either with or without a particular state.

This tool is particularly useful as new differentiation clusters and their associated antibodies are discovered.

The invention is industrially applicable in the field of tests for clinical immunology and oncology, in the field of cell-based tests for pharmaceutical research, and in the field of cell-based tests for fundamental research.

In addition to the above-mentioned tried-and-tested methods, the invention can also be used for many other applications.

In some embodiments, the invention is designed for cell phenotyping tests.

In one embodiment for phenotyping circulating lymphocytes, a CD3-targeting antibody may be deposited in a first zone and a CD4-targeting antibody in a second zone, a CD8-targeting antibody in a third zone, a CD19-targeting antibody in a fourth zone, and a CD16-targeting antibody mixed with a CD56-targeting antibody in a fifth zone.

In embodiments, the invention is designed to perform cell activation assays in response to molecular ligands.

In an embodiment for quantifying T lymphocyte activation, a CD3-targeting antibody can be deposited in admixture with a CD28-targeting antibody in a first zone, a CD4-targeting antibody in a second zone, a CD8-targeting antibody in a third zone and a CD69-targeting antibody in a fourth zone. In a variant of this embodiment, the antibody targeting CD69 can be replaced by an antibody targeting CD25, or a fifth zone can be added with the antibody targeting CD25.

In an embodiment for quantifying B cell activation, an antibody targeting the IgM Fc fragment and an antibody targeting CD69 can be deposited in a fourth zone.

In one embodiment for quantifying NK lymphocyte activation, an anti-CD16 antibody can be deposited in a first zone, an antibody targeting CD56 in a second zone, and an antibody targeting CD107 in a third zone.

In one embodiment for quantifying monocyte activation, an anti-CD16 antibody can be deposited in a first zone, an antibody targeting CD14 in a second zone, and an antibody targeting CD86 in a third zone. In a variant of this embodiment, the antibody targeting CD16 may be replaced by an antibody targeting CD32.

In one embodiment for quantifying the activation of chimeric antigen receptor T lymphocytes (CAR T-cells), a CD19 protein can be deposited in a first zone, and an antibody targeting CD69 in a second zone. In a variant of this embodiment, the antibody targeting CD69 may be replaced by an antibody targeting CD25.

In one embodiment, fibronectin can be deposited in a first zone, and an antibody targeting collagen in a second zone, to detect the fibrosing phenotype of fibroblasts. In one variant, TGF-β (TGF-beta) can be added to fibronectin in the first zone.

In some embodiments, the invention is designed to perform cell activation assays during intercellular interactions.

In an embodiment for the detection of antigen-specific T lymphocyte activation, an anti-CD14 antibody can be deposited in a first zone and an antibody targeting CD69 in a second zone. In a variant of this embodiment, the antibody targeting CD69 may be replaced by an antibody targeting CD25.

In one embodiment for detecting NK lymphocyte cytotoxicity, CD16, known to bind K562 target cells, can be deposited in a first zone and an antibody targeting CD107 in a second zone.

In one embodiment, a checkerboard of contiguous protein and antibody zones of different sizes and sub-cellular periods can be printed, and the single-cell measurement can be carried out straddling at least one protein zone and one antibody zone, by identifying the cell contour and analyzing adhesion in each zone present in the zone delimited by the contour.

Throughout the application, the words “subcellular dimensions” will refer to:
a zone smaller in length than the dimension of a cell of one type and of a cell of the same type activated to a particular state, or
a zone shorter in length than that of a zone straddling a cell of a first type and a cell of a second type, and not overlapping both cells when they are in contact. Typically, the size of a cell in suspension will be of the order of 8 μm and can extend to over 50 microns for a cell adhering to the substrate thanks to the invention.

In all embodiments, the invention can be completed by adding one or more zones each containing a protein targeting a membrane molecule or an antibody targeting a membrane molecule of a cell type selected by the zones of the embodiments described above.

### EXAMPLES

Protocols for implementing the teaching of the present application are presented below, by way of non-limiting examples.

### Application Example 1: Lymphocyte-Selective Substrates, without Substrate Activation and with Substrate Activation Readout

Protocol description:
a—surface treatment:
Under a dust-proof hood:
   Plasma-clean a 22×22 glass slide: Nexterion glass D Schott Minifab clean room cleaned (ref 1472309). Plasma cleaner pressure 300 mTor
   Put the plasma cleaner on High for 30 min.
   Remove the slides from the plasma and place each slide in a Petri dish.
   Make an APTS solution (200 μL/slide): in milli-Q water+1% (3-aminopropyl) triethoxysilane (APTS) (ref A3648 Sigma-Aldrich)+0.03% acetic acid 196 μL milli-Q water+2 μ 3% acetic acid+2 μL APTS
   Place the solution on the slides in a fume hood and incubate for 2 hrs at 4° C.
   Rinse the Petri dish 3 times with milli-Q water
   Dry the slide under nitrogen flow in the fume hood
   Dry on the hot plate at 95° C. for 15 min
   Use nitrogen to remove any dust
   Stick on the PDMS film (250 μm)
   Under the fume hood, glue the glass slide (UV glue) to the bottom of a perforated Petri dish (from the outside)
   Place the Petri dish in the UV cube (full surface, 1 min, 100% laser)
   Prepare the PEG-SVA solution (10 μL/well): milli-Q water, 10 mM carbonate buffer pH 8.5 and 23% PEG-SVA (mPEG-SUCCINIMIDY VALERATE MW 5000 Da, ref: 109681 INTERCHIM). For 40 μL of final solution: 36 μL of milli-Q water, 4 μL of 100 mM carbonate buffer and 0.0092 g of PEG SVA
   Add 10 μL to each well
   Incubate overnight at 4° C.
   Rinse 8 mL/well H2O milli-Q
b—Printing protocol
Markers (Pattern 1):
   BSA 4% 15 min at RT (20 μL/well)
   Rinse 8 mL/well at 4 mL/min
   Deposit 10 μL/well PLPP
   Print at 3000 mj/mm2 under oxygen flow
   Rinse 8 mL/well at 4 mL/min
   Deposit BSA-Fluorescein, incubate 5 min at RT
   Rinse PBS 8 mL/well at 4 mL/min
Passivation:
   PEG-SVA 5% 10 mM carbonate buffer 20 μL/well 5 min at RT
   Rinse 8 mL/well H2O milli-Q
   BSA 4% 20 μL/well 15 min at RT
   Rinse PBS 8 mL/well at 4 mL/min
Print 10 μm diameter center circles (Pattern 2):
   Deposit 10 μL/well PLPP
   Print at 3000 mj/mm2 under oxygen flow
   Rinse 8 mL/well at 4 mL/min
   Anti CD3-CD28 antibody deposition at 100 μg/mL-100 μg/mL-50 μg/mL respectively (final volume 20 μL/well) For one well: 2 μL CD3 and CD28, 1 μL CD45, 15 μL PBS
   Incubate overnight at 4° C.
   Rinse 8 mL/well at 4 mL/min
Passivation:
   PEG-SVA 5% 10 mM carbonate buffer 20 μL/well 5 min at RT
   Rinse 8 mL/well H2O milli-Q
   BSA 4% 20 μL/well 15 min at RT
   Rinse PBS 8 mL/well at 4 mL/min
Peripheral pattern printing (Pattern 3)
   Deposit 10 μL/well PLPP
   Print at 3000 mj/mm2 under oxygen flow
   Rinse 8 mL/well at 4 mL/min
   Anti CD69 antibody deposition at 100 μg/mL (final volume 20 μL/well) For one well: 4 μL CD69 and 16 μL PBS
   Incubate overnight at 4° C.
   Rinse 8 mL/well at 4 mL/min
Reference antibodies:
   Anti-hCD45 AB: ref NBP2-34804, clone SPM570, NovusBio
   Anti-hCD3 AB: Ultra leaf purified, ref 300438, clone UCHT1, Biolegend
   Anti-hCD28 AB: Ultra leaf purified, ref 302933, clone CD28.2, Biolegend
   Anti hCD69 AB: ref MAB2359, clone 298633, R&D system.

### Application Example 2: Substrates for Selecting and Identifying Memory T Lymphocytes, with Substrate Activation and Substrate Activation Readout

In this example, the same protocol will be followed as in Example 1, replacing CD3-CD28 with CD45RO.

### Application Example 3: T Lymphocyte Activation Measurements

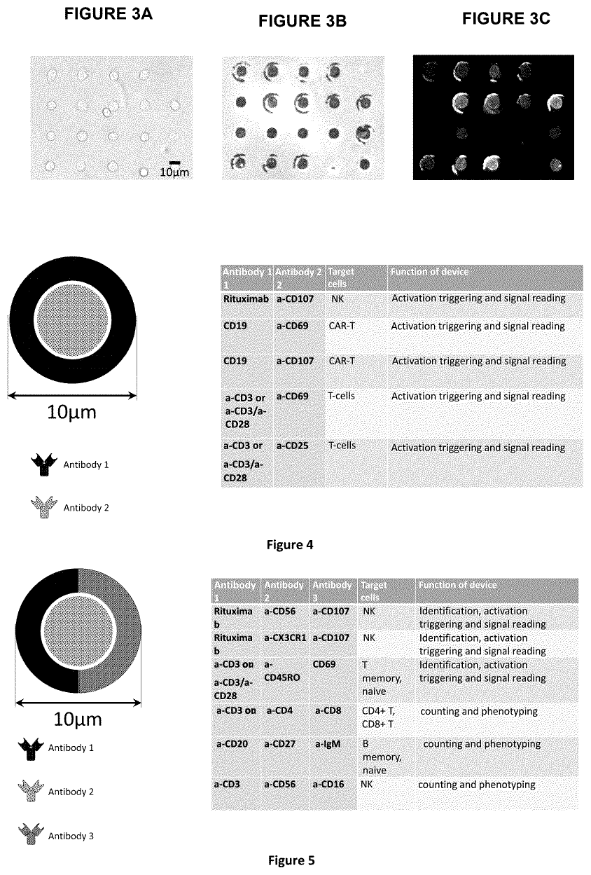

FIG. 3 shows an example of measurement in a device with micropatterns designed to accommodate single T lymphocytes and detect their activation by the TCR complex. These patterns were composed of two distinct zones, each with a specific function: the central zone contained anti-CD3 and anti-CD28 antibody to select and activate T lymphocytes, and the lateral zone contained anti-CD69 antibody to read the CD69 expression of activated cells. The interferometric reading is shown in FIG. 3B, at right. You can see that some anti-CD69 zones are dark, meaning that these captured T lymphocytes are activated in this experiment (confirmed by immunofluorescence). This example shows the concept of “smart” substrates with multiple antibodies, which is highly flexible and adaptable to different applications and cell types by choosing printed antibodies accordingly.

### Application Example 4: Other Possible Applications

The technology described in the present invention enables functional assays for personalized use of new immunotherapies by monitoring immune cell activation. Evaluating the efficacy of immunotherapies and stratifying patients as potential responders can optimize the use of these costly treatments. This is an important medical issue. Functional explorations are often too slow and complex to be translated into clinical contexts, where the costs of human handling and expertise are highly restrictive, and remain the preserve of academic research. The technology developed by the inventors of “smart” substrates with multiple functional micropatterns enables leukocyte activation to be measured in a matter of hours, and can effectively address an unmet need for the development of rapid, clinically accepted functional assays.

One of the main advantages of this technology is that it can be adapted to measure various functions of different leukocyte types. To adapt the technique to a new specific function and a new type of leukocyte, simply change the nature of the antibody in the micropatterns. The inventors have identified a (non-exhaustive) series of antibodies and molecules to be imprinted in order to perform the functions of selection/identification, activation triggering, and cell expression readout for various immune cells (Table 1). For most cell types and functions, models are based on antibody printing. In some cases, for example with CAR-T lymphocytes, it is important to imprint CD19 as the activation target, and CD19 is not an antibody. In all cases, adapting the technology to new applications relies on the same type of surface chemistry and physico-chemistry, and it's just a matter of changing the type of antibody in the already-proven preparation protocols to create new functional devices.

TABLE 1
Relevant antibodies and proteins for micropatterns capable of performing
specific functions (identification, selection, activation, stimulation,
response readout) with different cell types.

| | Identification/Selection | Activation | reading |
|---|---|---|---|
| Memory | α-CD45RO | α-CD3 | α-CD69 |
| T cells | | α-CD28 | α-CD107 |
| | | | α-CD25 |
| Naïve | α-CD45RA | α-CD3 | α-CD69 |
| T cells | | α-CD28 | α-CD107 |
| | | | α-CD25 |
| CAR | CD19 | CD19 | α-CD69 |
| T-cells | | | α-CD107 |
| | | | α-CD25 |
| | | | α-CD57 |
| | | | TIM-3 |
| | | | LAG-3 |
| NK cells | α-CD56 (+/−) | α-CD16 | α-CD107 |
| | α-CD3 (−) | Rituximab | |
| | | α-CD335 | |
| Monocytes | α-CD14 | α--CD16 | α-CD86 |
| | | α-CD32 | α-CD163 |
| | | α-CD64 | |
| | | LPS | |

For example, we may be interested in assessing the activation properties of CAR T-cells when engaging with a chimeric CD19 target, or of NK cells against a Rituximab target (see FIGS. 4 and 5).

In this example, glass slides (SCHOTT Nexterion) are plasma-treated for 15 minutes and topped with a PDMS molding (Polydimethylsiloxane, Sylgard 184) to create channels. A solution of APTES ((3-Aminopropyl)triethoxysilane, Sigma) diluted to 1% in milli-Q water with 0.03% acetic acid is incubated on the glass for 2 hrs at 4° C. The surface was then rinsed with water and incubated for 15 minutes at 95° C. A solution of PEG-SVA (MW: 5000 Da, INTERCHIM) in 10 mM carbonate buffer (NaHCO3) is then incubated on the glass for 12 hrs at 4° C. The channel is then rinsed with water and a solution of PLPP (15.5 mg·mL−1, Alveole) is introduced into the channels and stirred with a syringe pump (NEMESYS, Cetoni) at a flow rate of 4 μL×s⁻¹ throughout the exposure. The surface is exposed to a UV dose of 3,000 mJ×mm⁻² to create the binding pattern using the PRIMO platform (Alveole), then incubated with 50 μg·mL−1 of 6-x anti-His Tag (Life Technologies) for 1 hr at room temperature. The solution is then rinsed and the surface blocked for 15 minutes with 4% BSA (Bovin Serum Albumin, Sigma). A solution of CD19-His-tag (Life Technologies) at 20 μg×mL−1 is then incubated for 12 hrs at 4° C. The surface is then blocked again with 4% BSA for 20 minutes, before being re-insulated with UV light to create a peripheral pattern for activation reading. Finally, the surface is incubated for 1 hr at room temperature with human anti-CD69 (Bio Techne, R&D Systems). The cells are then introduced into the channel for 10 minutes, then rinsed so that only cells are left on the patterns. The reading is taken after 3 hrs of incubation of the cells in the device.

1. A device characterized in that it comprises a substrate comprising a first zone on which is adsorbed a protein capable of binding to a first membrane molecule and comprising a second zone on which an antibody is absorbed, targeting a second membrane molecule, the first zone and the second zone extending together in length over a dimension comparable to the length of a cell.

2. The device according to claim 1, wherein the first membrane molecule is a membrane molecule common to a first cell type and wherein the second membrane molecule is a membrane molecule common to a subtype of the first cell type.

3. The device according to claim 1, wherein the first membrane molecule is a membrane molecule common to a first cell type and wherein the second membrane molecule is a membrane molecule common to a particular type of the first cell type.

4. The device according to claim 1, wherein the first membrane molecule is a membrane molecule common to a first cell type and wherein the second membrane molecule is a membrane molecule common to a second cell type capable of interacting with the first cell type.

5. The device according to claim 1, wherein the protein is a CD4-targeting antibody and the antibody is a CD8-targeting antibody.

6. The device according to claim 1, wherein the protein is an antibody targeting CD3.

7. The device according to claim 1, wherein the protein is CD19.

8. The device according to claim 1, wherein the antibody is an antibody targeting CD69.

9. The device according to claim 1, wherein the antibody is an antibody targeting CD107.

10. The method characterized in that it comprises the following steps:
adsorbing an antibody capable of binding to a first membrane molecule, in a first subcellularly-dimensioned zone of the substrate,
adsorbing an antibody targeting a second membrane molecule onto a substrate, in a second subcellularly-dimensioned zone of the substrate.
